(19) United States
(12) Patent Application Publication
(10) Pub. No.: US 2012/0053409 A1
(43) Pub. Date: Mar. 1, 2012

(54) GUIDE ASSEMBLY FOR ENDOSCOPE

(76) Inventors: Shinichi Yamakawa, Kanagawa (JP); Takayuki Nakamura, Kanagawa (JP); Masayuki Iwasaka, Kanagawa (JP)

(21) Appl. No.: 13/193,204

(22) Filed: Jul. 28, 2011

(30) Foreign Application Priority Data
Aug. 25, 2010 (JP) 2010-187906

(51) Int. Cl. A61B 1/00

(52) U.S. Cl. 600/114

(57) ABSTRACT

A guide assembly of a self-propelled type for an endoscope having an elongated tube for entry in a body cavity is provided. An endless track device has an annular surface, for turning around endlessly on an endless track in an axial direction of a steering device of the elongated tube, and for propulsion by contacting a wall of the body cavity. A first driving device drives the endless track device. A second driving device is disposed on a proximal side from the first driving device, for receiving force applied by an external drive source, and driving the endless track device. A resilient connecting coil structure connects the first driving device to a distal side of the second driving device in the axial direction, and transmits the force from the second driving device to the first driving device, for actuation thereof in synchronism with one another.

### BACKGROUND OF THE INVENTION

1. Field of the Invention

The present invention relates to a guide assembly for an endoscope. More particularly, the present invention relates to a guide assembly capable of causing an endoscope to enter a body cavity smoothly, and keeping a steering device in the endoscope steerable readily even with a self-propelled structure for guiding.

2. Description Related to the Prior Art

An endoscope is used to diagnose a body cavity, such as a large intestine in a gastrointestinal tract. Manipulation of the endoscope is a difficult process, because the large intestine is a tortuous organ in a human body, and some body parts are very changeable in the position in the body, such as a sigmoid colon and a transverse colon. Learning the manipulation of the endoscope of the large intestine requires much experience and time. If a doctor is insufficiently skilled in the manipulation, physical load to the body of a patient will be very large.

U.S. Pat. Nos. 6,971,990 and 7,736,300 (corresponding to JP-A 2009-513250) disclose a self-propelled apparatus for propelling the endoscope in the axial direction in the body cavity to facilitate the manipulation even for an unskilled operator or doctor. The self-propelled apparatus of the documents includes a movable endless track device or crawler device or toroidal device. The endless track device is driven to turn around for the endoscope to travel mechanically. Force of propulsion is created by the endless track device contacting a wall of the large intestine, so as to guide the endoscope deeply in the body cavity.

However, U.S. Pat. Nos. 6,971,990 and 7,736,300 disclose the self-propelled apparatus in which a support or housing of the endless track device longitudinally extends in the axial direction of the elongated tube. There is a problem in that the steering of the steering device is obstructed by the combined use of the guide assembly or the self-propelled apparatus, and that flexibility of the elongated tube may be lower. Accordingly, the manipulation may be more difficult.

### SUMMARY OF THE INVENTION

In view of the foregoing problems, an object of the present invention is to provide a guide assembly capable of causing an endoscope to enter a body cavity smoothly, and keeping a steering device in the endoscope steerable readily even with a self-propelled structure for guiding.

In order to achieve the above and other objects and advantages of this invention, a guide assembly of a self-propelled type for an endoscope having an elongated tube for entry in a body cavity is provided, the elongated tube having a steering device on a distal side with a variable direction. An endless track device has an annular surface, for turning around endlessly in an axial direction of the steering device, and for propulsion by contacting a wall of the body cavity. First and second driving devices are mounted about the steering device, arranged with one another, for driving the endless track device in cooperation, the second driving device being disposed on a proximal side from the first driving device, for receiving force applied by an external drive source. A resilient connecting structure transmits the force from the second driving device to the first driving device, for actuation thereof in synchronism with one another.

The connecting structure includes at least two coils, having diameters different from one another, and combined by containing a first one in a second one thereof so that winding directions thereof are different from one another.

The first driving device is fixedly mounted on the steering device.

In one preferred embodiment, the first and second driving devices are fixedly mounted on the steering device.

Each of the first and second driving devices includes an engagement roller, having teeth, rotatable about an axis perpendicular to the axial direction, for moving the endless track device.

Furthermore, a flexible tubular cover covers the connecting structure.

The force is torque in a direction about an axis of the axial direction. Each of the first and second driving devices includes a worm gear sleeve, secured to the steering device, meshed with the engagement roller, rotated by the torque about the steering device, for rotating the engagement roller.

The connecting structure includes front and rear ends, the rear end is secured to the worm gear in the first driving device, and the front end is secured to the worm gear in the second driving device for rotation thereof.

Furthermore, a bearing sleeve is secured about the steering device, for supporting the worm gear in a rotatable manner about the axis.

Each of the first and second driving devices further includes a first ring sleeve disposed around the worm gear. A first through opening is formed in a wall of the first ring sleeve, for supporting the engagement roller in a rotatable manner. A second ring sleeve is disposed around the first ring sleeve, for supporting the endless track device movably. A second through opening is formed in a wall of the second ring sleeve. An idler roller is secured in the second through opening, for rotating about an axis perpendicular to the axial direction, and nipping the endless track device in cooperation with the engagement roller.

The idler roller is constituted by a pair of idler rollers, and the engagement roller is disposed between the idler rollers.

Furthermore, a regulating mechanism directs the endless track device away from the connecting structure by contact therewith between the first and second driving devices.

The regulating mechanism projects from a proximal end of the first driving device.

In another preferred embodiment, the regulating mechanism projects from a distal end of the second driving device.

The regulating mechanism includes a roller arm disposed to extend from the first or second driving device. A regulating roller is supported at an end of the roller arm in a rotatable manner, for pressing the endless track device.

The endless track device is formed from fluid-impermeable material, and internally charged with liquid.

In one preferred embodiment, the endless track device is formed from fluid-impermeable material, and internally charged with gel.

In another preferred embodiment, the endless track device is formed from biocompatible plastic material.

The second driving device is operated remotely.

Consequently, a steering device can be kept in the endoscope steerable readily even with a self-propelled structure for guiding, because of the two driving devices for an endless track device.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope system;

FIG. 2 is a perspective view illustrating a guide assembly;

FIG. 3 is an exploded perspective view illustrating the guide assembly;

FIG. 4 is a vertical section illustrating the guide assembly;

FIG. 5 is a vertical section illustrating another preferred guide assembly having regulating rollers.

### DETAILED DESCRIPTION OF THE PREFERRED EMBODIMENT (S) OF THE PRESENT INVENTION

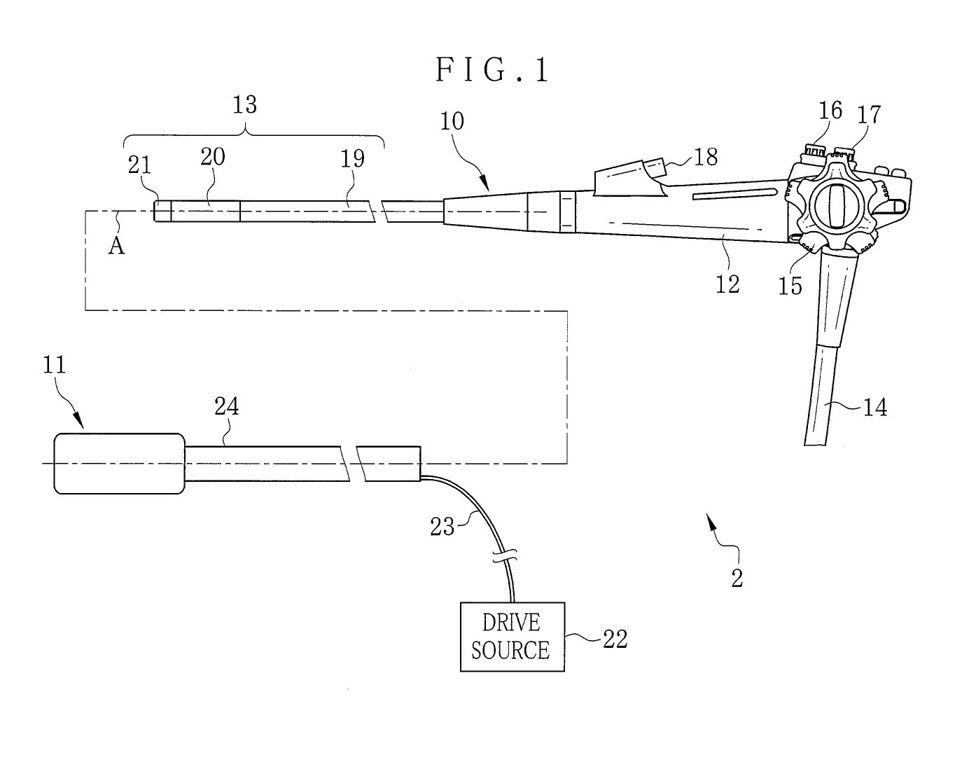

In FIG. 1, an endoscope system 2 includes an endoscope 10 and a self-propelled type of guide assembly 11. The endoscope 10 has a handle device 12 and an elongated tube 13 or guide tube disposed to extend from the handle device 12 for entry in a body cavity, for example, a large intestine of a gastrointestinal tract. A universal cable 14 is disposed to extend from the handle device 12. Connection plugs (not shown) are disposed at a proximal end of the universal cable 14 for connection with a light source apparatus and a processing apparatus (not shown) in a removal manner.

The handle device 12 includes steering wheels 15, an air/water button 16 and a suction button 17. The air/water button 16 is operable for supplying air or water through a distal end of the elongated tube 13. An instrument channel 18 is formed through the handle device 12 and the elongated tube 13 for receiving entry of a forceps, electrocautery device or other medical instrument.

The elongated tube 13 includes a flexible device 19, a steering device 20 and a head assembly 21 in a sequence in a distal direction from the handle device 12. The flexible device 19 has a length as great as several meters for reach of the head assembly 21 to an object of interest in a body cavity. The steering device 20 bends up and down and to the right and left in response to operation of the steering wheels 15 of the handle device 12. Thus, the head assembly 21 can be steered in a desired direction in the patient's body.

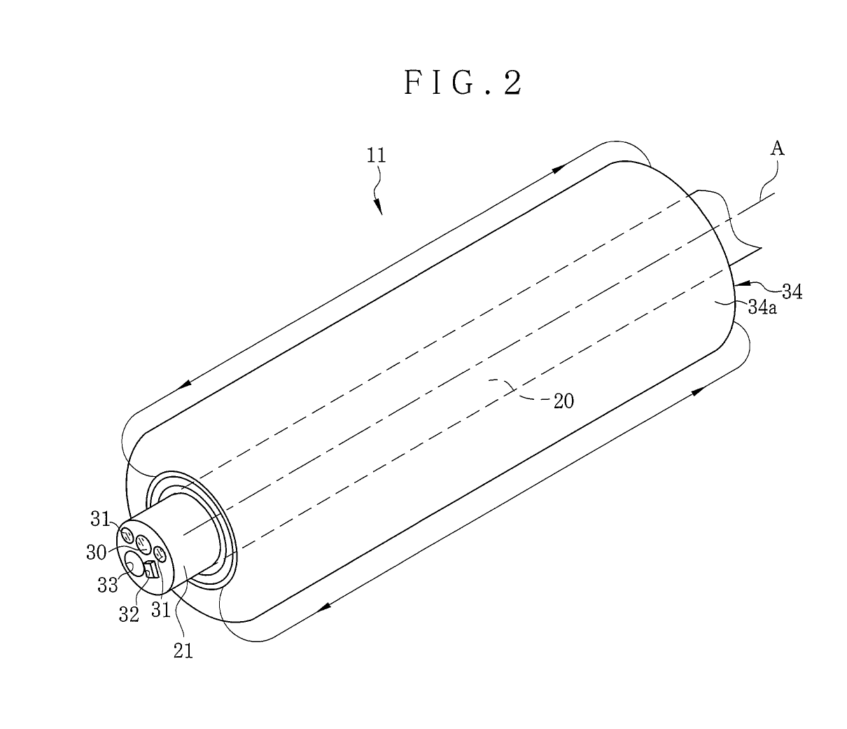

An imaging window 30 is formed in the head assembly 21 for imaging of a body part in the body. See FIG. 2. The head assembly 21 contains objective optics and an image sensor or solid-state image pickup device for imaging, such as CCD and CMOS image sensors. The image pickup device is connected to the processing apparatus by a signal line, which extends through the elongated tube 13, the handle device 12 and the universal cable 14. An object image of the body part is focused on a reception surface of the image pickup device, and is converted into an image signal. The processing apparatus processes the image signal from the image pickup device through the signal line by image processing, and obtains a video signal by conversion after the image processing. The object image is output and displayed on a monitor display panel (not shown) according to the video signal.

Various openings are formed in the head assembly 21 as illustrated in FIG. 2. Among those, a lighting window 31 passes illumination light from a light source apparatus toward an object of interest. An air/water nozzle 32 supplies air or water toward the imaging window 30 from an air/water supply device in the light source apparatus in response to depression of the air/water button 16. An instrument opening 33 causes a distal end of a medical instrument from the instrument channel 18 to appear distally.

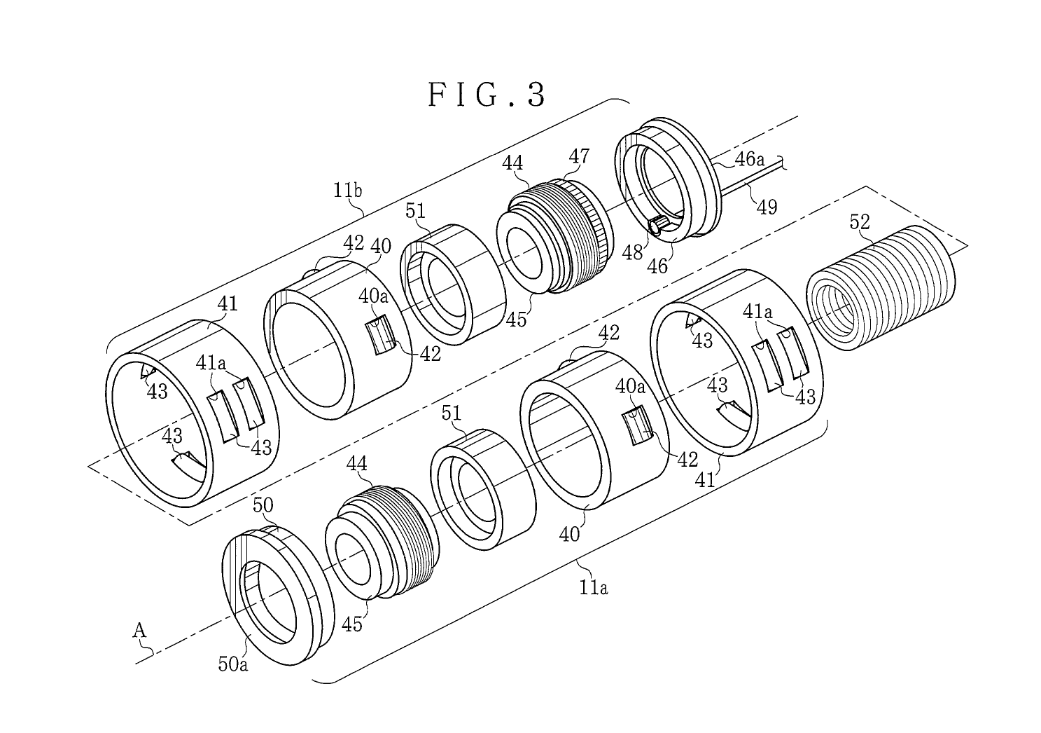

The guide assembly 11 is a device mounted on the endoscope 10, for assisting forward and backward movement of the elongated tube 13 of the endoscope 10 in a body cavity. There is an external drive source 22 which drives the guide assembly 11. A torque coil structure 49 of a multi component type or three component type is connected with an output shaft of the drive source 22. See FIG. 3. A protection sheath 23 receives entry of the torque coil structure 49 at its full length for covering. Torque of the drive source 22 is transmitted to the torque coil structure 49 to rotate in the protection sheath 23 as transmission device or wire device.

An overtube 24 is used to cover the elongated tube 13, and is ready to expand and shrink in an axial direction of an axis A of the elongated tube 13. The protection sheath 23 for the torque coil structure 49 is entered between the overtube 24 and the elongated tube 13.

A controller (not shown) controls the drive source 22. A button panel (not shown) is connected to the controller. The button panel includes a command button for inputting command signals for forward movement, backward movement and stop of the self-propelled type of guide assembly 11, and a speed button for changing a moving speed of the guide assembly 11. Note that a control program can be prepared suitably for an object to be imaged. The drive source 22 can be actuated according to the control program without manipulating the button panel, so as to actuate the guide assembly 11 automatically.

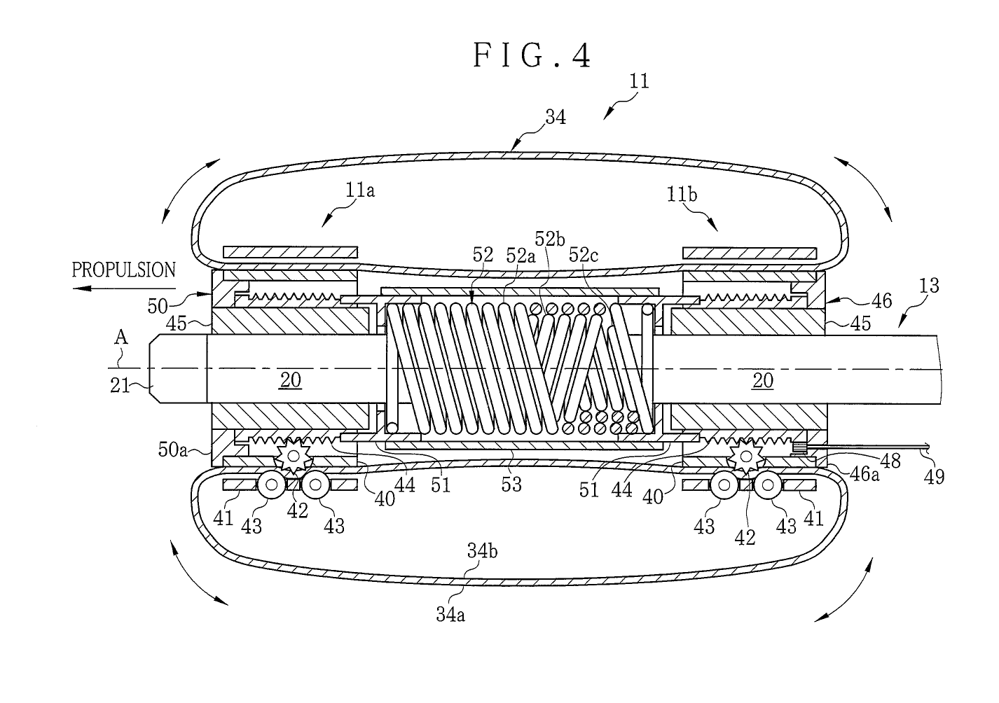

In FIG. 2, the guide assembly 11 includes a movable endless track device 34 or crawler device or toroidal device. The endless track device 34 has a hollow shape with an annular surface, is movable on an endless track, and is filled with liquid. See FIG. 4. The endless track device 34 is formed from a biocompatible plastic material having flexibility and fluid-impermeability. An example of the biocompatible plastic material is polyvinyl chloride. Also, polyamide resin, fluorocarbon resin, polyurethane resin and the like can be used. The inside of the endless track device 34 can be filled with suitable fluid, such as liquid, gel, gas, or a combination of at least two of liquid, gel and gas.

The endless track device 34 endlessly turns around in the axial direction of the axis A. When an outer surface 34a of the endless track device 34 contacts an inner wall of a body cavity, propelling force occurs to move the elongated tube 13 along the axis A. To propel the elongated tube 13 in the distal direction, the elongated tube 13 is moved by the turn around of the endless track device 34 in an endless manner to orient the outer surface 34a in the proximal direction. To move the elongated tube 13 in the proximal direction, the endless track device 34 is turned around endlessly to orient the outer surface 34a in the distal direction.

In FIGS. 3 and 4, the guide assembly 11 includes a first driving device 11a and a second driving device 11b or support devices with a rotating mechanism for self-propulsion. Those transmit the force from the drive source 22 to the endless track device 34, and are secured to the steering device 20 of the elongated tube 13 of the endoscope 10. The first driving device 11a is positioned on a distal side from the second driving device 11b.

Each of the driving devices 11a and 11b includes a first ring sleeve 40 and a second ring sleeve 41. Both of the first and second ring sleeves 40 and 41 are cylindrical, and have an equal size along the axis A. A diameter of the first ring sleeve 40 is smaller than that of the second ring sleeve 41. The first ring sleeve 40 is contained in and surrounded by the second ring sleeve 41. In FIG. 3, the endless track device 34 is not depicted.

Through openings 40a are formed in a wall of the first ring sleeve 40. An engagement roller 42 or drive roller or toothed roller has teeth, is disposed inside each of the through openings 40a, and rotates about an axis perpendicular to the axis A. The engagement roller 42 is disposed at the middle of the first ring sleeve 40 in the direction of the axis A. Three engagement rollers 42 are arranged at a pitch of 120 degrees in the circumferential direction of the first ring sleeve 40.

Through openings 41a are formed in a wall of the second ring sleeve 41. A pair of idler rollers 43 or driven rollers are disposed inside respectively the through openings 41a. Each of the idler rollers 43 is rotatable about an axis parallel to the axis of the engagement roller 42. Three pairs of idler rollers 43 are arranged at a pitch of 120 degrees in the circumferential direction of the second ring sleeve 41. When the first ring sleeve 40 is contained in the second ring sleeve 41, the second ring sleeve 41 is positioned relative to the first ring sleeve 40 to set the engagement roller 42 between the idler rollers 43. The endless track device 34 is mounted about the two second ring sleeves 41 by passage in their end openings. The endless track device 34 is nipped between the engagement roller 42 and the idler rollers 43. An inner surface 34b of the endless track device 34 is contacted by the idler rollers 43, which are rotated by endless turn around of the endless track device 34.

Specifically, the endless track device 34 is prepared in the following manner. At first, a plastic tube having two open ends with flexibility and elasticity is initially formed from a sheet or film of the above-described suitable material. The plastic tube is halfway inserted in a sleeve lumen of the two second ring sleeves 41. Then a portion of the plastic tube outside the sleeve lumen is bent back externally and extended to cover the periphery of the two second ring sleeves 41. A first side line of the inserted half of the plastic tube is opposed to a second side line of the bent half to the plastic tube, so that the halves are attached together along the first and second side lines by adhesion, welding or other suitable method. Finally, the toroidal shape of the endless track device 34 is obtained.

A worm gear 44 or worm thread is contained in the first ring sleeve 40. The worm gear 44 is included in a worm gear sleeve (worm drive or worm sleeve). A bearing sleeve 45 or holding sleeve supports the worm gear 44. The elongated tube 13 of the endoscope 10 is entered in a center hole of the bearing sleeve 45, and tightly fitted therein. The head assembly 21 projects distally from the bearing sleeve 45 upon mounting the bearing sleeve 45 on the elongated tube 13. A worm thread of the worm gear 44 rotates about the bearing sleeve 45 along the axis A. The worm gear 44 is meshed with the engagement roller 42, which is rotated by the worm gear 44.

A rear end ring 46 is attached to the first ring sleeve 40 of the second driving device 11b. A flange 46a is a portion of the rear end ring 46 at its peripheral edge. The flange 46a, when the rear end ring 46 is attached to the first ring sleeve 40, contacts a rear edge of the first ring sleeve 40. The bearing sleeve 45 is fitted in an inner hole of the rear end ring 46 in a tight manner without dropping.

Spur gear teeth 47 or a driven gear is formed with a proximal end of the worm gear 44 in the second driving device 11b, the teeth being arranged about the axis A. A pinion 48 is disposed on the rear end ring 46 in a rotatable manner. An axis of the pinion 48 is parallel to the axis A. The pinion 48 is secured to an end of the torque coil structure 49. Thus, the pinion 48 is rotated together with the torque coil structure 49. The torque coil structure 49 includes three coil springs combined in a multi layer form in such a state that their winding directions are different from one another. The torque coil structure 49 can transmit torque even upon rotating in any of the forward and backward directions. When the pinion 48 rotates, the spur gear teeth 47 rotate responsively.

A front end ring 50 is attached to the first ring sleeve 40 of the first driving device 11a. A flange 50a is a portion of the front end ring 50 at its peripheral edge. The flange 50a, when the front end ring 50 is attached to the first ring sleeve 40, contacts a front edge of the first ring sleeve 40. An end of the bearing sleeve 45 is fitted in an inner hole of the front end ring 50 in a tight manner without dropping.

A proximal end of the worm gear 44 in the first driving device 11a has an outer annular recess. A distal end of the worm gear 44 in the second driving device 11b has an outer annular recess. A connecting ring 51 is fitted in each outer annular recess of the worm gear 44 on a suitable side. There is a connecting coil structure 52 of a multi component type or three component type. Each of ends of the connecting coil structure 52 is fitted on the connecting ring 51. The connecting ring 51 and the connecting coil structure 52 rotate together with the worm gear 44. Thus, torque of the worm gear 44 of the second driving device 11b is transmitted to the worm gear 44 of the first driving device 11a.

The connecting coil structure 52 includes a first coil spring 52a, a second coil spring 52b and a third coil spring 52c. The first coil spring 52a is positioned externally. The second coil spring 52b has an outer diameter substantially equal to an inner diameter of the first coil spring 52a. The third coil spring 52c has an outer diameter substantially equal to an inner diameter of the second coil spring 52b. The coil springs 52a, 52b and 52c are combined in a multi layer form in such a state that their winding directions are different from one another. Specifically, the first and third coil springs 52a and 52c have turns wound in the counterclockwise direction. The second coil spring 52b has turns wound in the clockwise direction.

When the connecting coil structure 52 is rotated in the counterclockwise direction, the first and third coil springs 52a and 52c are shifted and tightened in an inward direction, the second coil spring 52b being shifted and loosened in an outward direction. Thus, torque can be transmitted with high efficiency owing to the tight contact between the first and second coil springs 52a and 52b. When the connecting coil structure 52 is rotated in the clockwise direction, the first and third coil springs 52a and 52c are shifted and loosened in the outward direction, the second coil spring 52b being shifted and tightened in the inward direction. Thus, torque can be transmitted with high efficiency owing to the tight contact between the second and third coil springs 52b and 52c. Note that a structure and operation of the torque coil structure 49 are basically the same as the connecting coil structure 52.

A tubular cover 53 is flexible along the axis A of the elongated tube 13, and has one end to which the connecting ring 51 is secured. The tubular cover 53 covers the connecting coil structure 52, and prevents body fluid from contacting the connecting coil structure 52. In FIG. 3, the tubular cover 53 is not depicted.

The operation of the endoscope system 2 is described now. At first, the overtube 24 is retained on the elongated tube 13 of the endoscope 10. The elongated tube 13 is entered in the bearing sleeve 45 to mount the guide assembly 11 on the elongated tube 13.

After securing the overtube 24 and the guide assembly 11 to the endoscope 10, a power source of the processing apparatus, light source apparatus and controller is turned on. Then personal information of the patient is input. The elongated tube 13 of the endoscope 10 is entered in his of her body cavity.

After the head assembly 21 is advanced to a predetermined body part, for example, slightly short of a sigmoid colon, then the button panel is operated to turn on a power source for the drive source 22 of the self-propelled type of guide assembly 11. Then a command signal for start is input with the button panel. The drive source 22 rotates the torque coil structure 49 in a predetermined direction. In the second driving device 11b, the pinion 48 is rotated by rotation of the torque coil structure 49. The worm gear 44 is rotated by the pinion 48 discretely. The connecting coil structure 52 rotates in response to rotation of the worm gear 44 in the second driving device 11b, to rotate the worm gear 44 in the first driving device 11a. In the embodiment, torque of the worm gear 44 in the second driving device 11b is transmitted by the connecting coil structure 52, so as to rotate the worm gear 44 in the first driving device 11a with the torque efficiently.

When the worm gear 44 rotates together with the driving devices 11a and 11b, the engagement roller 42 is rotated responsively. Thus, the endless track device 34 endlessly turns around. The guide assembly 11 advances in the axial direction of the elongated tube 13 when the outer surface 34a of the endless track device 34 contacts a wall of a body cavity. Consequently, the head assembly 21 advances along the wall of the body cavity.

When a command signal for a change is input by operating the button panel, the drive source 22 changes a rotational speed of the torque coil structure 49. Thus, a moving speed of the self-propelled type of guide assembly 11 is changed. When a command signal for return is input by operating the button panel, the drive source 22 causes the torque coil structure 49 to rotate in a backward direction, to move the guide assembly 11 and the head assembly 21 backwards. When a command signal for a stop is input by operating the button panel, the drive source 22 stops to stop moving the guide assembly 11. It is possible to propel the head assembly 21 through the body cavity to an object of interest by suitably repeating those steps of the movement.

A doctor or operator steers the steering device 20 of the endoscope 10 by manipulating the steering wheels 15, to bend the head assembly 21 in a desired direction. As the second driving device 11b is connected to the first driving device 11a by the connecting coil structure 52 in the guide assembly 11, the connecting coil structure 52 bends together with the steering device 20 according to the flexibility, for the driving devices 11a and 11b to follow the steering of the steering device 20. This is effective in keeping the steering device 20 free from being obstructed by the guide assembly 11.

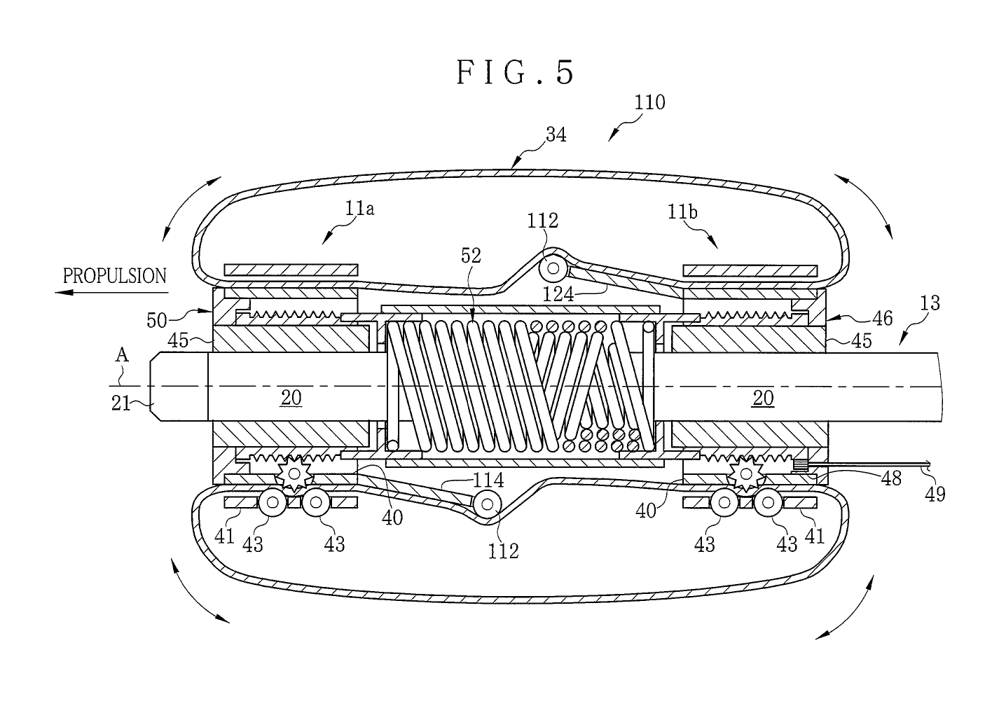

The feature of the invention with the two driving devices for the endless track device 34 may be combined with additional structures. In FIG. 5, another preferred self-propelled type of guide assembly 110 is illustrated. A regulating roller 112 or contact roller as a regulating mechanism prevents the endless track device 34 from flexing inwards between the driving devices 11a and 11b, to prevent the endless track device 34 from contacting the worm gear 44 or the tubular cover 53 of the connecting coil structure 52. Elements similar to those of the above embodiment are designated with identical reference numerals.

In FIG. 5, a roller arm 114 or roller support in the guide assembly 110 projects from a proximal end of the first ring sleeve 40 of the first driving device 11a to extend toward the second driving device 11b. The regulating roller 112 is supported by an end of the roller arm 114. A roller arm 124 or roller support in the guide assembly 110 projects from a distal end of the first ring sleeve 40 of the second driving device 11b to extend toward the first driving device 11a. The regulating roller 112 is supported by an end of the roller arm 124. Note that the number and arrangement of the roller arm 114 or 124 may be changed suitably for the purpose.

For example, a set of four regulating rollers can be preferably used and arranged at a pitch of 90 degrees in correspondence with the steerable structure of the steering device up and down and to the right and left.

In the embodiment, the connecting coil structure 52 is used between the two worm gears 44 in the driving devices 11a and 11b. However, other elements may be used between the two and having flexibility along the axis A, for example, only one coil spring, a rubber tube or the like.

Although the driving devices 11a and 11b are disclosed in the above embodiment, the number of the driving devices for self-propulsion may be three or more. Also, the number of the drive sources or motors for the driving devices for self-propulsion may be one or three or more.

In the above embodiment, the bearing sleeve 45 is used for each of the first and second driving devices. However, elements other than the bearing sleeve 45 can be used. At least one of the first and second driving devices should be fixedly mounted on the elongated tube. A predetermined one of those can have a bore substantially equal to an outer diameter of the elongated tube for fixed mounting. The remaining one of those can have a bore sufficiently larger than the outer diameter of the elongated tube for a rotatable or slidable state.

Note that, in the course of assembly of parts for the guide assembly 11, the two second ring sleeves 41 are exactly positioned inside the endless track device 34. The second ring sleeves 41 with the endless track device 34 can be held manually by an operator of production, or automatically by a producing machine.

In the above embodiments, the self-propelled type of guide assembly is used with the endoscope for a medical use. Also, the guide assembly of the invention can be used with an endoscope for industrial use, an ultrasonic probe, or other instruments for imaging in a cavity.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly of a self-propelled type for an endoscope having an elongated tube for entry in a body cavity, said elongated tube having a steering device on a distal side with a variable direction, comprising:
an endless track device, having an annular surface, for turning around endlessly in an axial direction of said steering device, and for propulsion by contacting a wall of said body cavity;
first and second driving devices, mounted about said steering device, arranged with one another, for driving said endless track device in cooperation, said second driving device being disposed on a proximal side from said first driving device, for receiving force applied by an external drive source;
a resilient connecting structure for transmitting said force from said second driving device to said first driving device, for actuation thereof in synchronism with one another.

2. A guide assembly as defined in claim 1, wherein said connecting structure includes at least two coils, having diameters different from one another, and combined by containing a first one in a second one thereof so that winding directions thereof are different from one another.

3. A guide assembly as defined in claim 2, wherein said first driving device is fixedly mounted on said steering device.

4. A guide assembly as defined in claim 2, wherein said first and second driving devices are fixedly mounted on said steering device.

5. A guide assembly as defined in claim 3, wherein each of said first and second driving devices includes an engagement roller, having teeth, rotatable about a perpendicular axis perpendicular to said axial direction, for moving said endless track device.

6. A guide assembly as defined in claim 3, further comprising a flexible tubular cover for covering said connecting structure.

7. A guide assembly as defined in claim 5, wherein said force is torque exerted in a direction about an axis extending in said axial direction;
each of said first and second driving devices includes a worm gear sleeve, secured to said steering device, meshed with said engagement roller, rotated by said torque about said steering device, for rotating said engagement roller.

8. A guide assembly as defined in claim 7, wherein said connecting structure includes front and rear ends, said rear end is secured to said worm gear sleeve in said first driving device, and said front end is secured to said worm gear sleeve in said second driving device for rotation thereof.

9. A guide assembly as defined in claim 8, further comprising a bearing sleeve, secured about said steering device, for supporting said worm gear sleeve in a rotatable manner about said perpendicular axis.

10. A guide assembly as defined in claim 8, wherein each of said first and second driving devices further includes:
a first ring sleeve disposed around said worm gear sleeve;
a first through opening, formed in a wall of said first ring sleeve, for supporting said engagement roller in a rotatable manner;
a second ring sleeve, disposed around said first ring sleeve, for supporting said endless track device movably;
a second through opening, formed in a wall of said second ring sleeve;
a first roller, secured in said second through opening, for rotating about said perpendicular axis, and nipping said endless track device in cooperation with said engagement roller.

11. A guide assembly as defined in claim 10, wherein said first roller is constituted by a pair of first rollers, and said engagement roller is disposed between said first rollers.

12. A guide assembly as defined in claim 3, further comprising a regulating mechanism for directing said endless track device away from said connecting structure by contact therewith between said first and second driving devices.

13. A guide assembly as defined in claim 12, wherein said regulating mechanism projects from a proximal end of said first driving device.

14. A guide assembly as defined in claim 12, wherein said regulating mechanism projects from a distal end of said second driving device.

15. A guide assembly as defined in claim 12, wherein said regulating mechanism includes:
a roller arm disposed to extend from said first or second driving device;
a regulating roller, supported at an end of said roller arm in a rotatable manner, for pressing said endless track device.

16. A guide assembly as defined in claim 3, wherein said endless track device is formed from fluid-impermeable material, and internally charged with liquid.

17. A guide assembly as defined in claim 3, wherein said endless track device is formed from fluid-impermeable material, and internally charged with gel.

18. A guide assembly as defined in claim 3, wherein said endless track device is formed from biocompatible plastic material.

19. A guide assembly as defined in claim 3, wherein said second driving device is operated remotely.
